(19) United States
(12) Patent Application Publication
(10) Pub. No.: US 2025/0302343 A1
(43) Pub. Date: Oct. 2, 2025

(54) AN ELECTRODE ARRAY FOR MEASURING THE PH OF ANIMAL TISSUES, A PROBE COMPRISING SUCH AN ARRAY, AND AN ASSEMBLY COMPRISING SAID PROBE

(71) Applicant: QUANTUM INNOVATIONS SP. Z O.O., Warszawa (PL)

(72) Inventors: Grzegorz SUWALSKI, Warszawa (PL); Lukasz GÓRSKI, Warszawa (PL); Przemyslaw FURDAL, Warszawa (PL); Elzbieta MALINOWSKA, Warszawa (PL)

(21) Appl. No.: 18/881,759

(22) PCT Filed: Jul. 7, 2023
(86) PCT No.: PCT/PL2023/050053

§ 371 Date: Jan. 7, 2025

(30) Foreign Application Priority Data
Jul. 7, 2022 (PL) P.441669

(51) Int. Cl. A61B 5/145; A61B 5/00; A61B 5/01; A61B 5/1473

(52) U.S. Cl. CPC A61B 5/14539; A61B 5/01; A61B 5/14735; A61B 5/6848; A61B 5/6879; A61B 2560/0468; A61B 2562/0271; A61B 2562/063; A61B 2562/125; A61B 2562/227

(57) ABSTRACT

The present invention relates to an electrode array (1) for measuring the pH of animal tissues comprising an indicator electrode (2), and a reference electrode (3). The reference electrode (3) is all solid state and comprises a steel wire (3a) with an applied layer of conductive polymer (3b) and with a membrane (3c) containing ionic liquid and polyurethane. Furthermore, the present invention relates to a probe (11) for real-time continuous measurement of the pH of animal tissues comprising a body (12) in the form of a mounting capsule in which an electrode array (1) according to the present invention, a vacuum system (13), a temperature sensor (14) are housed. The vacuum system (13) is designed to attach the probe (11) to the tissue surface and comprises a vacuum tube (13a) and a vacuum surface (13b), wherein the temperature sensor (14) is a contact sensor designed to be applied to the surface of the tissue.

The present invention relates to an electrode array for measuring the pH of animal tissues.

More specifically, the present invention relates to a probe for real-time continuous measurement of the pH of animal tissues comprising said electrode array and an assembly comprising said probe.

### TECHNICAL FIELD

Monitoring the pH of tissue allows appropriate therapeutic decisions to be made in many medical procedures. This is particularly the case in cardiac surgery, vascular surgery, or transplantology.

During such procedures, tissue acidemia, i.e. a reduction in pH below the physiological value (e.g. for the heart muscle, this is a pH value below about 6.8), can occur due to tissue ischemia. As a consequence of such ischemia, irreversible damage and necrosis may occur.

Tissue acidemia is a parameter indicating the predominance of dangerous anaerobic metabolism in cells, as the product of this process is largely lactic acid, the concentration thereof in the tissue leads to a lower pH. This acidemia is a proven strong marker of increased risk of perioperative mortality and dangerous complications in patients undergoing medical procedures.

Cardiac surgery can lead to myocardial acidemia.

Most cardiac surgery requires cardiac arrest. Only then is it possible to open it and perform the planned surgical procedures, such as e.g. heart valve replacement. Moreover, cardiac arrest ensures a stationary surgical field, which is necessary for some operations on the heart's surface. The temporary exclusion of the heart from the patient's natural circulation also results in a stoppage of blood flow through the coronary arteries which consequently activates anaerobic metabolism causing local acidification of the heart muscle. This acidification can be monitored using the pH of the heart muscle tissue. In the surgical situation, to avoid the activation of pathological anaerobic metabolism, a cardioplegic solution is injected directly into the coronary arteries, which cools the heart, stops its electrical and mechanical activity, and drastically slows down the metabolism of the heart cells, thus temporarily preventing them from dying. A situation in which cardiac protection with a cardioplegic solution is ineffective during a cardiac procedure leads to a return of cardiac cell metabolism while the oxygen supply is lacking. Insufficient cardiac protection leading to acidemia and intraoperative damage to the heart is one of the main causes of mortality and the occurrence of complications after cardiac surgery.

Monitoring pH values is also important for other tissues, primarily muscle. Monitoring the pH of muscle tissue in patients with acute ischemia of the lower or upper limbs, for example in the case of acute aortic dissection, allows the acidification of the ischemic tissue to be identified and therapeutic decisions to be made (re-surgery, another procedure or, in irreversible cases, the need for amputation). Monitoring pH levels can therefore immediately allow for an assessment of whether there is an improvement in the blood supply to the tissue and whether the acidemia is eliminated by surgery to create a new blood supply to the relevant tissue groups or body parts, or by replantation surgery (re-suturing of an amputated body part).

Monitoring pH levels is also relevant in transplantology. The success of organ transplants depends on their proper protection (adequate stopping of metabolism) during transport. Ineffective protection results in early failure of the transplanted organ and manifests itself in organ acidemia.

### BACKGROUND ART

Electrode arrays for measuring the pH of animal tissues are known.

EP1503661 discloses a pH tissue monitoring system. The system comprises a first electrode disposed in an anterior wall of a ventricle and a second electrode disposed in the posterior wall of said ventricle. A known and commercially available silver chloride electrode is used. The system is disposed in a probe that can be delivered to a site within the human body using a catheter and/or endoscope. The electrodes are sutured to the inside of the tissue.

FR2744804 discloses a probe for measuring the potential difference between two measuring locations corresponding to the tissue of a human or animal organ. The probe comprises two electrodes. One of these electrodes is in the form of a needle which is pressed into the tissue. The other electrode (a reference electrode) is covered by a elastically deformable porous material and has the shape of a plate and is attached to the tissue surface using a thread.

U.S. Pat. No. 10,321,861 discloses a method of diagnosis using a pH sensor to estimate the condition of soft tissues in vivo e.g. muscle, fat, composite cell and tissue cultures (explants), and cell cultures both grown in vitro. Moreover, the pH sensor can be used for medical diagnosis of organs e.g. heart, lung, and kidney. The sensor incorporates the well-known and commercially available silver chloride electrode as a measurement/reference electrode.

U.S. Pat. No. 8,095,196 discloses a sensor probe for in-situ measurement of pH in a human tissue (e.g., cardiovascular) environment. This probe comprises a needle, disposed in the tissue environment, and an optic cable. The pH measurement is based on the measurement of changes in light excitation, which is transmitted down the optical fiber.

### SUMMARY OF INVENTION

Therefore, there is a need to provide an electrode array that will allow real-time monitoring of animal tissue pH using electromotive force measurement, while the array will meet the requirements of biocompatibility, and mechanical resistance and will be independent of the chloride concentration of the tissue being monitored.

Moreover, there is a need to provide a probe for real-time monitoring of the pH of animal tissues, wherein the pH of a precisely defined area is measured and the probe itself is attached to the tissue surface in a non-invasive manner, i.e. without additional disruption to the tissue's consistency.

Moreover, there is also a need to provide an assembly comprising a probe for real-time monitoring of animal tissue pH, a monitor, and a calibration solution, the assembly being able to indicate the absolute pH value of the monitored tissue.

The above purposes are achieved by the electrode array according to claim 1, the probe according to claim 16, and the assembly according to claim 25. Preferred embodiments are the object of the dependent claims.

The object of the present invention is the electrode array for measuring the pH of animal tissues comprising an indicator electrode, and a reference electrode, the reference electrode being all solid state and comprising a steel wire with an applied layer of conductive polymer and with a membrane containing ionic liquid and polyurethane.

Preferably, the object is the electrode array according to the present invention characterized in that the indicator electrode is an antimony electrode, a ruthenium (IV) oxide electrode, or an electrode with a conductive polymer and polymer membrane.

Preferably, the object is the electrode array according to the present invention characterized in that the indicator electrode is the antimony electrode deposited on a steel wire using a galvanostatic technique.

Preferably, the object is the electrode array according to the present invention characterized in that the indicator electrode is an electrode with a deposited conductive polymer and polymer membrane layer comprising a pH-sensitive ionophore, wherein the conductive polymer is deposited on a steel wire using a galvanostatic technique and polymer membrane is deposited with using dip-coating technique.

Preferably, the object is the electrode array according to the present invention characterized in that the conductive polymer in the reference electrode can be selected from PEDOT: PSS [poly(3,4-ethylenedioxythiophenc)-poly(styrenesulfonate)], polypyrrole or polyaniline.

Preferably, the object is the electrode array according to the present invention characterized in that the conductive polymer in the reference electrode is PEDOT: PSS [poly(3,4-ethylenedioxythiophene)-poly(styrenesulfonate)].

Preferably, the object is the electrode array according to the present invention characterized in that the conductive polymer in the reference electrode is applied to the steel wire by electropolymerization or a ready-made suspension with the conductive polymer.

Preferably, the object is the electrode array according to the present invention characterized in that the reference electrode membrane contains from 1% to 5% ionic liquid in polyurethane.

Preferably, the object is the electrode array according to the present invention characterized in that the reference electrode membrane contains 2% ionic liquid in polyurethane.

Preferably, the object is the electrode array according to the present invention characterized in that the ionic liquid in the reference electrode membrane is selected from the group consisting of 1-ethyl-3-methylimidazolium bis(trifluoromethylsulfonyl)imide, 1-butyl-3-methylimidazolium hexafluorophosphate, 1-butyl-3-methylimidazolium acetate, 1-butyl-3-methylimidazolium chloride, choline acetate and/or choline phosphate.

Preferably, the object is the electrode array according to the present invention characterized in that the ionic liquid in the reference electrode membrane is 1-ethyl-3-methylimidazolium bis(trifluoromethylsulfonyl)imide.

Preferably, the object is the electrode array according to the present invention characterized in that the reference electrode membrane contains 4 mg of 1-ethyl-3-methylimidazolium bis(trifluoromethylsulfonyl)imide and 196 mg of polyurethane.

Preferably, the object is the electrode array according to the present invention characterized in that the reference electrode membrane contains either plasticized polyurethane or unplasticized polyurethane.

Preferably, the object is the electrode array according to the present invention characterized in that the reference electrode and the indicator electrode are in the form of a wire or a needle.

Preferably, the object is the electrode array according to the present invention characterized in that the reference electrode and the indicator electrode have a diameter in the range of 0.2 to 1.5 mm.

Preferably, the object is the electrode array according to the present invention characterized in that the reference electrode and the indicator electrode have a diameter of 0.5 mm.

The object of the present invention is also a probe for real-time continuous pH measurement of animal tissues comprising a body in the form of a mounting capsule in which the electrode array according to the present invention, a vacuum system, a temperature sensor is housed, the vacuum system being designed to attach the probe to the tissue surface and comprising a vacuum tube and a vacuum surface, the temperature sensor being a contact sensor designed to be applied to the surface of the tissue.

Preferably, the object is the probe according to the present invention characterized in that the temperature sensor is applied to the external surface of the tissue.

Preferably, the object is the probe according to the present invention characterized in that the probe at its periphery at the point of adhesion to the tissue comprises a suction cup envelope.

Preferably, the object is the probe according to the present invention characterized in that the probe comprises a signal cable and a display device, the signal cable connecting the indicator electrode, the reference electrode, and the temperature sensor to the display device for the pH measurement result.

Preferably, the object is the probe according to the present invention characterized in that the display device is a monitor.

Preferably, the object is the probe according to the present invention characterized in that the probe body is made of a material selected from medical silicones, acrylonitrile-butadiene-styrene terpolymer (ABS), HDPE, LDPE, polypropylene, PETG, poly-carbonate, polyester, Kydex, POM, acetal copolymer, Delrin®, PET-P, Fluorosint®, Halar®, Hydex (PBT-P), Kynar, Noryl, Nylon, PEEK, polyethylenes (including LDPE, HDPE, and UHMW), polypropylene homopolymer, PPSU, PSU, Radel® A, Radel® R, Rulon 641, composites and mixtures thereof.

Preferably, the object is the probe according to the present invention characterized in that the body of the probe is made of acrylonitrile-butadiene-styrene terpolymer.

Preferably, the object is the probe according to the present invention characterized in that the probe has a lower body part and an upper body part, the lower body part comprising a passage and a cable path for the temperature sensor, a passage and a cable path for the indicator electrode, and a passage and a cable path for the reference electrode, a lead-through for the signal cable and the latch tabs, and the upper part of the body comprises a clamp for the signal cable and the latch grooves, the latch tabs of the lower part and the latch grooves of the upper part being the latching mechanism.

Preferably, the object is the probe according to the present invention characterized in that the probe has a cylindrical shape.

The object of the present invention is also an assembly for measuring pH comprising the probe according to the present invention, a display device, and a calibration solution.

In view of the invention, it is possible to precisely monitor in real-time the pH of a specific area of animal tissue, also taking into account the effect of temperature, with the measurement being independent of the chloride concentration in the controlled area. Many prior art solutions have used a glass electrode as the indicator electrode, but its low mechanical resistance increases the risk of damage to the electrode during measurement. On the other hand, a silver chloride electrode without its electrolyte (the so-called pseudo-reference electrode), which is often used as the reference electrode, is sensitive to chloride ions, the concentration of which can change during the measurement (e.g. the cardioplegic solution contains KCl). The disadvantage of the previously used silver chloride electrodes is also the interfering effect of chloride ions, which are present and constantly changing their local concentration in the tissues, on the pH measurement. The invention also enables the probe to be attached to the tissue non-invasively, i.e. without additional damage to the tissue, eliminating mechanical attachment means (e.g. using thread). Thus, it enables easy and quick placement of the probe (on the tissue) and precise pH measurement and eliminates the risk of bleeding. In view of the invention, the pH measurement takes place each time at a predetermined tissue depth.

### BRIEF DESCRIPTION OF DRAWINGS

The invention will now be described with reference to the accompanying drawings, in which

FIG. 1 shows the results of mechanical resistance testing of the indicator electrode made of antimony-coated surgical steel; FIG. 1a shows the dynamic response of the electrodes before insertion into the tissue sample, FIG. 1b shows the dynamic response of the electrodes after insertion into the tissue sample, FIG. 1c shows the electrode calibration curves before insertion into the tissue sample, FIG. 1d shows the electrode calibration curves after insertion into the tissue sample.

FIG. 2 shows changes in the potential of the electrode array according to the present invention illustrated with graphics from consecutive stages of acid diffusion in the gelatin block.

FIG. 3 shows the course of potential changes recorded for a cell consisting of an antimony indicator electrode and the reference electrode with a polymer membrane containing the ionic liquid bis(trifluoromethyl sulfonyl)imide 1-ethyl-3-methylimidazolium.

FIG. 4 schematically shows the electrode array according to the present invention.

FIG. 5 schematically shows the pH probe according to the present invention.

FIG. 6 schematically shows the pH probe according to the present invention in the “top” view.

FIG. 7 shows the pH probe according to the present invention in an unfolded state with the division into the lower part of the body and the upper part of the body.

FIG. 8 shows the pH probe according to the present invention in a folded state.

FIG. 9 shows a schematic diagram of the method of protection and subsequent detection of acidification in the human heart muscle.

FIG. 10 schematically shows the pH probe according to the present invention attached to the surface of the heart.

### DESCRIPTION OF EMBODIMENTS

Only the details necessary to understand the invention are shown in the figures. Constructions and details which are not essential to the understanding of the invention but are obvious to one skilled in the art have been omitted from the figures for the purpose of emphasizing only the characteristics of the invention.

### Electrode Array

An electrode array for measuring the pH of animal tissues according to the present invention comprises:
an indicator electrode and
a reference electrode.

### Indicator Electrode

The indicator electrode is a stainless steel wire with deposited antimony, ruthenium (IV) oxide, or a conductive polymer and polymer membrane layer Preferably the indicator electrode is an electrode made of stainless steel, e.g. SS 316L surgical steel, with antimony deposited or with a conductive polymer deposited and a polymer membrane layer. A polymer membrane layer in an electrode with a conductive polymer deposited and a polymer membrane layer comprises a pH-sensitive ionophore.

Deposition of antimony, ruthenium (IV) oxide, or a conductive polymer on the indicator electrode can be performed either by a galvanostatic technique or by a galvanodynamic technique. Preferably, the deposition is performed by the galvanostatic technique. A layer of a polymer membrane of an electrode with a conductive polymer and a polymer membrane layer is deposited using dip-coating technique.

The steel wire in the indicator electrode can have a diameter ranging from 0.2 to 1.5 mm. Preferably, the diameter is 0.5 mm, which provides a good compromise between mechanical resistance and low invasiveness when driven into tissue.

The indicator electrode is either in the form of a wire or a needle.

The indicator electrode (i.e. its active part-the part that measures) has a length in the range of 0.1 to 0.9 cm. Preferably, the length is in the range of 0.5 to 0.6 cm. This is related to the depth of insertion of the electrodes into the tissue, such as heart muscle tissue. By inserting the electrodes to such a depth, an optimal pH measurement is obtained, in particular a pH measurement of the heart muscle. The length of the electrode is related to the expected depth of insertion into the tissue and can vary depending on the tissue being tested.

### Production of Indicator Electrodes

The antimony indicator electrode was made according to the following procedure.

An antimony deposition solution was prepared to contain:
potassium antimony tartrate K2Sb2(C4H206)2·3 H2O (1.002 g);
KCl (0.134 g);
HCl concentration-was dropped in with stirring until the precipitate dissolved (approx. 5 ml);
H2O (20 ml).

The stainless steel wire (0.5 mm diameter, 6 cm length) (SS 316L surgical steel) was degreased with acetone. Before antimony deposition, the steel was prepared by electrochemical etching in a 38 g/l NaOH solution (galvanostatic process, anodic current of 0.0216 A for 120 s) and etching in a 10% solution of H2SO4 (15 min). Antimony layers were deposited on such a substrate. The following electrochemical deposition parameters were used: galvanostatic deposition: cathodic current 0.00152 A, deposition time of 120 s or 240 s.

Deposition at 120 s gave a very adherent antimony layer, whereas at longer deposition times the antimony layer was too thick and had poor adhesion to the steel. All electrodes had slopes of calibration curves in the range 45-47 mV/pH in the pH range 4-8. Repeatability of the potential of electrodes produced under the same conditions in the 5 mV range was observed. Potential oscillations in the range below 1 mV were evident.

A ruthenium(IV) oxide indicator electrode was made according to the following procedure.

Wire pieces (0.5 mm diameter, 6 cm length) of stainless steel (SS 316L surgical steel) were degreased with acetone. Before ruthenium deposition, the steel was prepared by electrochemical etching in a 38 g/l NaOH solution (galvanostatic process, anodic current of 0.0216 A for 120 s) and etching in a 10% solution of H2SO4 (15 min). RuO2 layers were deposited on such a substrate. The following deposition methods were used:
Cyclic voltammetry (CV): −0.7 V to 0.5 V potential, 0.05 V/s, 15 cycles;
potentiostatic deposition: potential −0.6 V, 180 s;
galvanostatic deposition: cathodic current 0.01 A, 300 s.

Moreover, a nickel adhesion layer was prepared on part of the electrodes, before ruthenium deposition: a solution of 4.8 g NiCl2.6H20, 1,66 ml HCl, 20 ml water. It was deposited galvanostatically (cathodic current 0.016 or 0.01 A, 300 s).

Deposition using the CV technique did not provide the expected results, while potentiostatic deposition and galvanostatic deposition gave positive results. The electrodes are characterized by the slope of the calibration curves in the range of 52-56 mV/pH in the pH range of 4-8. For electrodes with a galvanostatic deposition layer, a better convergence of potentials was observed between individual specimens (potential spread of approximately 8 mV).

Electrodes with a nickel adhesion layer show potential drift approximately 4-5 minutes after immersion in solution. Electrodes without this layer are characterized by a stable potential.

An electrode with a conductive polymer and a polymer membrane was made according to the following procedure.

Wire pieces (0.5 mm diameter, 6 cm length) of SS 316L surgical steel were degreased with acetone and electrochemically deposited with a PEDOT: PSS conductive polymer layer. Electropolymerization was performed in an aqueous solution containing 0.1 M NaPSS and 0.01 M PEDOT. Galvanostatic deposition, anodic current of 0.0785 milliamperes for 714 s. Subsequently, a membrane of the following composition: H+I ionophore (Hydrogen ionophore I, Merck Cat. No. 95292), 198 mg of PU dissolved in 1.5 ml THF and 0.5 mg of ionic additive being potassium tetrakis [3,5-bis(trifluoromethyl)phenyl] borate was applied twice to the electrode by dip coating (with an interval of 15 min). As other components of the membrane a polyether based thermoplastic polyurethane (e.g. Tecoflex EG-80A, supplied from Lubrizol) are used, e.g.. After drying, the electrodes were conditioned for 8 hours in PBS solution pH 7.4.

Electrodes with a conductive polymer and a polymer membrane are calibrated using conventional solutions (e.g. PBS). Tests confirm that electrodes prepared in such a way provide pH measurement sensitivity suitable for the present invention.

### Testing the Mechanical Resistance of the Antimony Electrode

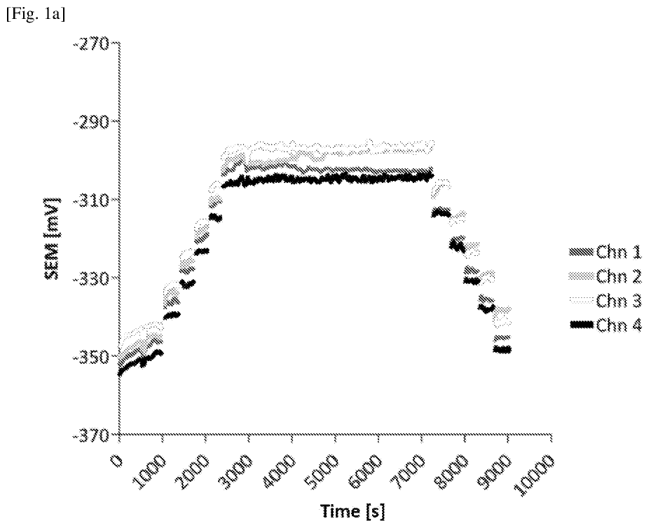

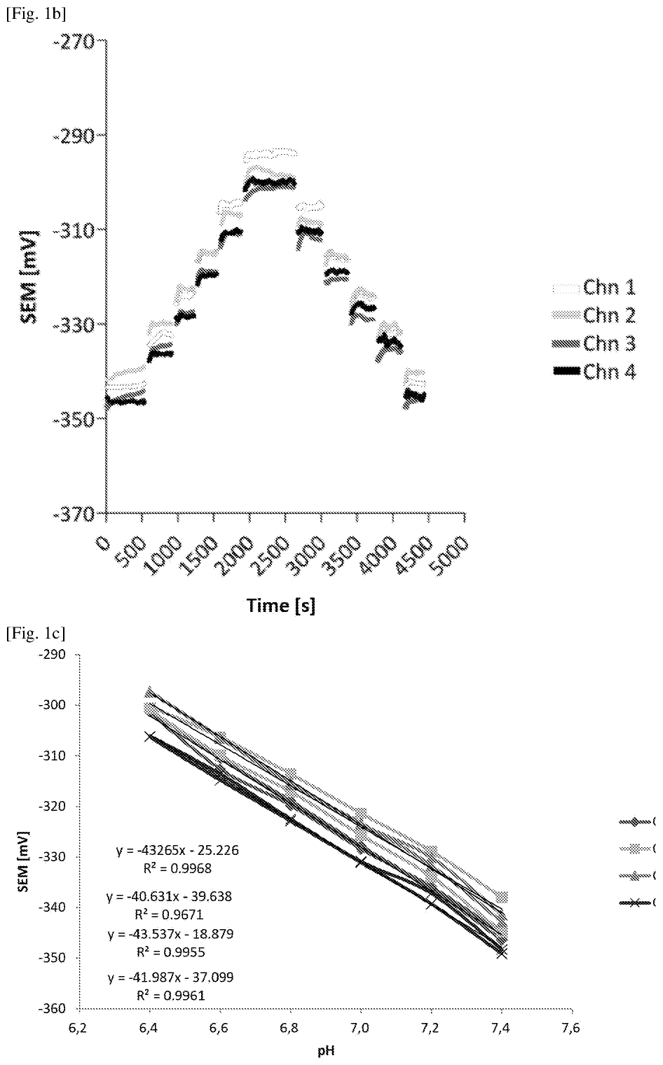

The four electrodes were calibrated to a pH ranging between 7.4 and 6.4 using PBS solutions (in 0.2 pH unit increments). Each electrode was then driven five times into the animal tissue sample, which was a chicken breast, to a depth of approximately 5 mm. The electrodes were rinsed with distilled water from a sprinkler and recalibrated in the same way. The graphs show the dynamic response ([FIG. 1a] and [FIG. 1b]) and calibration curves ([FIG. 1c] and [FIG. 1d]) for pH electrodes made of antimony-coated steel wire, before ([FIG. 1a], [FIG. 1c]) and after ([FIG. 1b], [FIG. 1d]) the electrodes were driven five times into the chicken breast tissue sample. Both the sensitivity of the pH measurement and its reversibility did not change significantly. A comparison of the results shows that the tested electrodes are not damaged when driven into meat, which models their driving into, for example, heart tissue ([FIG. 1a-d]).

### Optimum Adhesion Achievement of the Antimony Layer and its Mechanical Abrasion Resistance

To improve the adhesion of the antimony layer to the steel wire, deposition at a lower current density was tested. For this purpose, wire portions (0.5 mm diameter, 6 cm length) of SS 316L surgical steel were degreased and prepared by electrochemical etching according to the above-disclosed procedure. The electrochemical deposition parameters used were selected to maintain the electrical charge magnitude used in the previously described deposition processes at a lower current density. Electrochemical parameters: galvanostatic deposition: cathode current 0.00076 A, deposition time 240 s.

The obtained electrodes were characterized by slopes of calibration curves in the range of 46-47 mV/pH and pH of 7.4-6.4, but some hysteresis of the response was observed depending on the direction of pH changes. Also, the adhesion of the antimony layer was weaker than for electrodes obtained at higher current densities. Therefore, the previously developed antimony deposition parameters should be considered optimal.

### Optimized Structure of the Antimony Indicator Electrode

SS 316 surgical steel wire, 5 mm long and 0.5 mm in diameter (active part), degreased by washing with acetone and electrochemical etching in a 38 g/l NaOH solution (galvanostatic process, anodic current of 0.00432 A for 120 s). Antimony deposition was performed from solution:
Potassium antimony tartrate K2Sb2(C4H206)2·3 H2O (1.002 g);
KCl (0.134 g);
HCl concentration (approx. 5 ml);
H2O (20 ml).

Electrochemical parameters of the deposition process: cathode current 0.000304 A, deposition time 120 s.

### Conclusions

During the tests, the ruthenium oxide indicator electrode was found to have good pH sensitivity; however, the RuO2 layer adhered poorly to the stainless steel. Attempts were made to improve the adhesion by using an adhesion layer in the form of electroplated nickel. This did not produce the desired result. Also, further attempts to optimize the deposition process, i.e. changing: the deposition technique used (galvanostatic and galvanodynamic deposition), the current density, and the composition of the electroplating solution, did not yield the desired results.

The antimony indicator electrode did not cause problems with the adhesion of the pH-sensitive layer to the stainless steel. Antimony deposition was performed using both galvanostatic and galvanodynamic techniques; however, galvanostatically produced electrodes were characterized by preferred electrochemical parameters. Hence, as the indicator electrode, an electrode made of steel wire on which antimony was deposited using the galvanostatic technique is optimal.

The reference electrodes with a conductive polymer and a polymer membrane exhibited high mechanical resistance and good adhesion to the stainless steel wire. The deposition of a conductive polymer was performed using both galvanostatic and galvanodynamic techniques. Galvanostatically produced electrodes were preferred in terms of electrochemical performance. Hence, as the indicator electrode with a conductive polymer and a polymer membrane, an electrode made of steel wire on which a conductive polymer has been deposited using the galvanostatic technique and a polymer membrane has been deposited using the dip-coating, is optimal. Such an electrode comprises a pH-sensitive ionophore and a layer of conductive polymer. Depending on the structure, an electrode with a conductive polymer and a polymer membrane may also contain a plasticizer, e.g. dioctyl sebacate.

Reference electrode

The reference electrode is an all-solid-state electrode. The reference electrode is a stainless steel wire, e.g. surgical steel SS 316L, with an applied layer of conductive polymer and a membrane containing ionic liquid and polyurethane. The conductive polymer in the reference electrode may be selected from PEDOT: PSS [poly(3,4-cthylenedioxythiophene)-poly(styrenesulfonate)], polypyrrole, or polyaniline. Preferably, the conductive polymer in the reference electrode is PEDOT: PSS [poly(3,4-cthylenedioxythiophene)-poly(styrenesulfonate)].

Preferably, the conductive polymer in the reference electrode is applied to the steel wire using electropolymerization or a ready-made suspension with the conductive polymer. More preferably, the conductive polymer in the reference electrode is applied to the steel wire by electropolymerization.

Preferably, the reference electrode membrane contains from 1% to 5% ionic liquid in polyurethane. More preferably, the reference electrode membrane contains 2% ionic liquid in polyurethane.

Preferably, the ionic liquid in the reference electrode membrane is selected from the group consisting of 1-ethyl-3-methylimidazolium bis(trifluoromethylsulfonyl)imide, 1-butyl-3-methylimidazolium hexafluorophosphate, 1-butyl-3-methylimidazolium acetate, 1-butyl-3-methylimidazolium chloride, choline acetate and/or choline phosphate. More preferably, the ionic liquid in the reference electrode membrane is 1-ethyl-3-methylimidazolium bis(trifluoromethylsulfonyl)imide.

Preferably, the reference electrode membrane contains 4 mg of bis(trifluoromethyl sulfonyl)imide 1-ethyl-3-methylimidazolium and 196 mg of polyurethane dissolved in 1.5 ml of THF.

The reference electrode membrane contains either plasticized polyurethane or unplasticized polyurethane. Preferably, polyurethane is unplasticized.

The reference electrode is either in the form of a wire or a needle.

The reference electrode has a length in the range of 0.1 to 0.9 cm (its active part). Preferably, the length is in the range of 0.5 to 0.6 cm. This is related to the depth of insertion of the electrodes into the tissue, such as heart muscle tissue. By inserting the electrodes to such a depth, an optimal pH measurement is obtained, in particular a pH measurement of the heart muscle. The length of the electrode is related to the expected depth of insertion into the tissue and can be adjusted accordingly depending on the tested tissue.

### Production of Reference Electrodes

The reference electrode with PEDOT: PSS was made according to the following procedure. Wire portions (0.5 mm diameter, 6 cm length) of SS 316L surgical steel were degreased with acetone and coated with a layer of the conductive polymer PEDOT: PSS in the form of a commercial suspension (2.8 wt % dispersion in water, Sigma Aldrich, USA, product no.: 560596). After drying (30 minutes), a membrane composed of 15 mg ionic liquid, 92.5 mg PU, and 92.5 mg o-nitrophenyl octyl ether (plasticizer) dissolved in 1.5 ml THF. After drying, the electrodes were conditioned for 16 h in a solution of 10-3 M NaCl. Thus made electrodes had an average potential drift of about 1 mV/hour in a 12-hour measurement.

Achieving optimal membrane adhesion and mechanical resistance to abrasion, i.e. reducing the risk of the membrane remaining in the heart tissue

### Removal of Plasticizer

To improve the mechanical resistance of the reference electrodes, membranes were made from a polyurethane matrix without a plasticizer. Previously, a PU/plasticizer (o-nitrophenyl octyl ether) matrix in a 1:1 mass ratio was used, but such membranes tended to detach from the steel wire and stick together during storage.

Moreover, for better adhesion, the steel wire was degreased with acetone and electrochemically cleaned-electrochemical etching in a 38 g/l NaOH solution, anodic current of 0.0216 A for 120 s, and etching in a 10% solution of H2SO4 for 15 min.

A layer of conductive polymer PEDOT: PSS in the form of a suspension was applied to the substrate and thus prepared. After drying (30 min), a membrane containing 4 mg ionic liquid and 196 mg PU, was dissolved in 1.5 ml THF. After drying, the electrodes were conditioned for 16 hours in PBS solution, pH 7.4.

The adhesion of the membrane to the steel substrate and the mechanical properties of such electrodes were significantly improved. No peeling of the membranes from the wire (which prevented the electrodes from working) or sticking of the electrodes to each other during storage (which led to damage of the membranes when attempts were made to separate them) were observed.

### Thicker Membrane Layer

Reference electrodes were coated with a polymer membrane by dipping a wire modified with a layer of conductive polymer into a solution of the membrane components (dip-coating technique). During the testing of such electrodes, it was found that damage to such a membrane was common, resulting in high potential drift. To improve the mechanical resistance of the reference electrodes, electrodes with a thicker membrane were prepared. The steel wire was degreased with acetone and electrochemically cleaned in NaOH solution. A layer of conductive polymer PEDOT: PSS was electrochemically applied to the substrate and thus prepared. Subsequently, a membrane containing: 4 mg ionic liquid and 196 mg PU, dissolved in 1.5 ml THF, was applied to the electrode four times (with an interval of 15 min). After drying, the electrodes were conditioned for 16 h in PBS solution, pH 7.4. The potential stability of these electrodes was then tested in the same solution for 8 h.

For the electrodes thus prepared, the average drift at this time was 0.64 mV/h. Taking into account the theoretical value of the slope of the calibration curve on pH (59.2 mV/pH unit) and the expected maximum application time of the device (approximately 2-4 h), the measured drift of the reference electrode potential should be considered small and not affecting the pH measurement.

### Repeatable Production Achievement With the Maintained Convergence of Potentials Between Arrays

This feature was achieved by changing the application technique of conductive polymer. The conductive polymer layer (PEDOT/PSS) was applied using an electropolymerization technique (previously, it was applied from a suspension, which was an obstacle to ensuring reproducibility).

Electropolymerization was performed on purified stainless steel wires in an aqueous solution comprising 0.1 M NaPSS and 0.01 M EDOT. Galvanostatic deposition, anodic current of 0.0785 milliamperes for 714 s. The electrodes thus prepared were conditioned in PBS, pH 7.4 for 8 hours.

The electrodes were subjected to potential stability tests of the reference electrodes in PBS solutions with a pH range of 7.4 to 6.4 and again to 7.4 (changing every 0.2 pH unit). There was better convergence of potentials between the specimens, with electrode potentials within the 15 mV range. Importantly, the potentials of the electrodes remained in the same range when inserted into the chicken breast tissue sample, simulating the anticipated use of the device.

Potential stability achievement with up to 1.5 mV/h potential drift over 8 hours.

The electrodes made in the above manner were tested for potential stability. Experiments were performed on buffers over a period of 2 and 8 hours. The made electrodes had an average potential drift of approximately 1.5 mV/hour when measured over 8 hours.

Measurement capability maintenance despite membrane damage.

The potential was then measured in PBS solutions of pH 7.4 and 6.4 before and after mechanical damage to the membrane. The membrane was damaged in two ways: first by firmly squeezing the electrode (about 3 mm from the end) with fluted tweezers, and then by scratching the membrane three times with a blade.

The tested electrode arrays maintained significantly better potential stability compared to previously tested electrodes with a layer of conductive polymer applied from a suspension. These electrodes are more mechanically resistant due to better adhesion to the substrate of the conductive polymer layer applied by electropolymerization.

A minimum effective amount of ionic liquid as ensuring greater biocompatibility, and reduction of toxic effects on cardiac cells.

The effect of the amount of ionic liquid in the polymer membrane was tested. For this purpose, membranes comprising 1%, 2%, 3%, or 5% of the ionic liquid in a matrix consisting of polyurethane for the remainder were prepared. The tests were performed in classical microelectrodes (Electrode Body ISE, Merck, cat. no. 45137), which gives greater reproducibility of the results. The results of the tests indicated slight differences in the potential stability of the tested electrodes, so it was decided to consider the composition with the least amount of ionic liquid—2%—as the optimum, due to the lowest cost and the highest probability of biocompatibility and risk of negative effects on cardiac cells.

Biocompatibility achievement with maintained measurement capability by selecting the ionic liquid.

An evaluation of the acute cytotoxicity of the reference electrode components was performed. The obtained results indicated that electrodes comprising ionic liquids selected from the group: 1-ethyl-3-methylimidazolium bis(trifluoromethyl sulfonyl)imide, 1-butyl-3-methylimidazolium hexafluorophosphate, 1-butyl-3-methylimidazolium acetate, 1-butyl-3-methylimidazolium chloride, choline acetate, choline phosphate exhibited no significant toxicity.

Due to its best potentiometric properties (good potential stability in PBS test solutions with pH in the range of 7.4 to 6.4 and at a constant pH of 7.4 for 8 h) and lowest cytotoxicity, 1-ethyl-3-methylimidazolium bis(trifluoromethyl sulfonyl)imide was specified as the ionic liquid for the reference electrode.

Cytotoxicity-based biocompatibility tests were performed according to the procedures of ISO 10993-5:2009 (E) and ISO 10993-12:2021 (E) standards.

### Optimal Reference Electrode Structure

SS 316 surgical steel wire, 5 mm long and 0.5 mm in diameter (active part), degreased by washing with acetone and electrochemical etching in 38 g/L NaOH solution (galvanostatic process, anodic current of 0.00432 A for 120 s). Deposition of the PEDOT: PSS transition layer, an aqueous solution comprising 0.1 M NaPSS and 0.01 M EDOT. Galvanostatic deposition, anodic current of 0.0157 mA for 700 s. Subsequently, a membrane containing: 4 mg of ionic liquid (1-ethyl-3-methylimidazolium bis (trifluoromethylsulfonyl) imide) and 196 mg of PU dissolved in 1.5 ml of THF is applied to the electrode four times (by immersion). After THE drying, the electrodes were conditioned for 16 h in PBS solution, pH 7.4.

### Conclusions

During the tests, it was found that the most convenient choice would be the reference electrode comprising a polymer membrane containing an ionic liquid that forms a homogeneous solution with the polymer matrix.

Polyurethane was identified as the polymer matrix. Polyurethane exhibited better adhesion to solid substrates and biocompatibility than previously known matrices (e.g. plasticized poly (vinyl chloride)).

The possibility of using membranes without the addition of a plasticizer was also tested. Unexpectedly, it was found that for membranes without plasticizers, the potential stability was satisfactory, and therefore a preferred variant of the invention involves the use of an unplasticized membrane made of polyurethane.

The possibility of using an intermediate layer between the membrane and the transducer (steel wire), in the form of a conductive polymer layer, in the reference electrode was also tested. Unexpectedly, it was found that inserting a layer of PEDOT: PSS conductive polymer between the membrane containing the ionic liquid and the steel wire provided excellent electrode performance.

Both electropolymerization of the polymer and the application of a commercially available polymer suspension were used. Better potential stability was observed for electrodes with a conductive polymer transition layer applied by electropolymerization.

The amount of ionic liquid introduced into the polymer membrane of the reference electrode was also found to be an essential parameter. A higher amount of this compound improves the stability of the electrode potential, but this reduces the biocompatibility. Membranes containing 1%, 2%, 3%, or 5% ionic liquid in the polyurethane membrane were tested. A content of 2% ionic liquid in the membrane was found to sufficiently stabilize the electrode potential, so this amount was determined to be optimal.

To improve the biocompatibility of the electrodes, membranes comprising 1-ethyl-3-methylimidazolium bis(trifluoromethylsulfonyl)imide, 1-butyl-3-methylimidazolium hexafluorophosphate, 1-butyl-3-methylimidazolium acetate, 1-butyl-3-methylimidazolium chloride, choline acetate or choline phosphate were made. Of these, 1-ethyl-3-methylimidazolium bis(trifluoromethylsulfonyl)imide and 1-butyl-3-methylimidazolium acetate were the best to stabilize the reference electrode potential. The biocompatibility tests concluded that 1-ethyl-3-methylimidazolium bis(trifluoromethylsulfonyl)imide would be the optimal choice.

### Electrode Array Structure

To demonstrate the effect of the electrode array according to the present invention, experiments were performed with a gelatin block. A gelatin solution was prepared to measure the potential change: 5 g gelatin, 0.9 g NaCl, 5 cm3 saturated bromothymol blue solution, and 100 cm3 water. The solution was brought to pH 7.2 using NaOH. The complete solution was heated in a water bath to dissolve the gelatin. The solution was poured into 24 mm diameter glass rings and placed in the refrigerator to concentrate.

The measurements used antimony electrodes obtained at a deposition time of 120 s or electrodes with a polymer membrane and reference electrodes with a membrane composition of 15 mg ionic liquid, 92.5 mg PU. The electrodes were driven into the block from the top to a depth of approximately 5 mm. The block was placed on a Petri dish into which a 0.1 M solution of H3PO4 was poured.

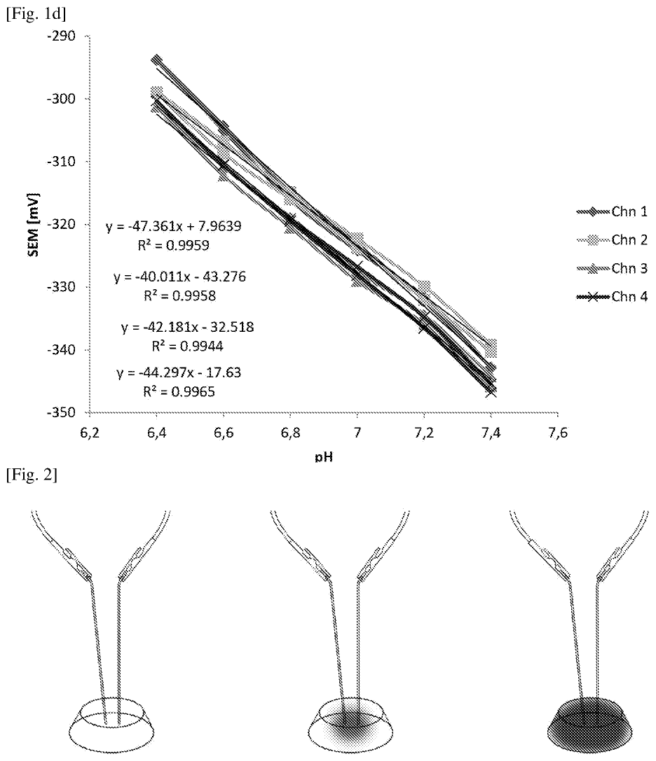

Diffusion of the acid in the block was observed by the change in color of the bromothymol blue from green to yellow (in [FIG. 2] visible by the gradual darkening of the block).

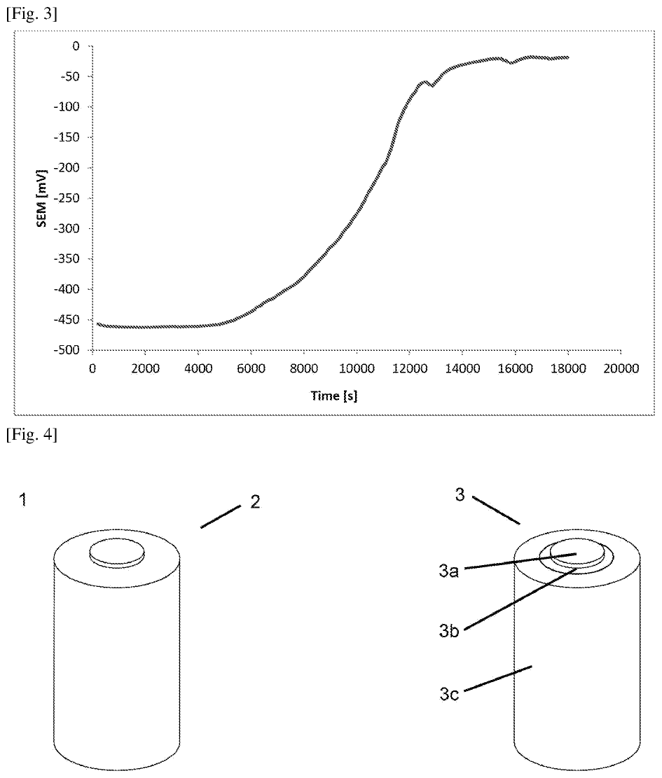

The course of the potential change is shown in the graph in [FIG. 3]. The increase in electromotive force recorded for the cell consisting of the antimony indicator electrode and the reference electrode with a polymer membrane containing the ionic liquid bis(trifluoromethylsulfonyl)imide 1-ethyl-3-methylimidazolium demonstrates the correct operation of the electrode array.

Analogous results in terms of potential changes are also obtained when a polymer membrane electrode is used instead of the antimony electrode as the indicator electrode.

The experiment confirmed that the electrode array according to the present invention has the properties to measure the electromotive force of the samples and thus to determine the pH value.

The electrode array 1 for measuring the pH of animal tissues according to the variant embodiment of the invention shown in [FIG. 4] comprises the indicator electrode 2 and the reference electrode 3.

The reference electrode 3 is all solid-state and comprises a steel wire 3a with an applied layer of conductive polymer 3b and a membrane 3c comprising ionic liquid and polyurethane.

In a preferred variant embodiment, the electrode array 1 comprises the indicator electrode 2 and the reference electrode 3. The indicator electrode 2 is a wire of SS 316L surgical steel with a deposited antimony layer. The indicator electrode 2 has a diameter of 0.5 mm and is in the form of a needle. The antimony layer is deposited using a galvanostatic technique. The reference electrode 3 is a wire 3a of SS 316L surgical steel with a deposited layer of conductive polymer 3b, which is PEDOT: PSS [Poly(3,4-ethylenedioxythiophene)-poly(styrenesulfonate)] and with a membrane 3c containing the ionic liquid bis (trifluoromethylsulfonyl) imide 1-ethyl-3-methylimidazolium (4 mg) and polyurethane (196 mg). Polyurethane is an unplasticized polyurethane. The reference electrode 3 has a diameter of 0.5 mm and is in the form of a needle. A layer of conductive polymer 3b and ionic liquid membrane 3c is deposited using an electropolymerization technique.

Other possible example embodiments of the invention are described below.

In the example embodiment, the indicator electrode 2 can be a ruthenium (IV) oxide electrode.

In the example embodiment, the indicator electrode 2 can be an electrode with a conductive polymer and a polymer membrane. Such a polymer membrane electrode comprises a pH-sensitive ionophore in a polymer membrane

In the example embodiment, the conductive polymer 3b in the reference electrode 3 is applied to a steel wire 3a using a ready-made suspension with conductive polymer 3b.

In the example embodiment, the indicator electrode 2 and the reference electrode 3 can be coated with a substance that prevents platelets from clinging to the electrodes, preferably Nafion® may be such a substance.

In the example embodiment, the membrane 3c of the reference electrode 3 comprises 1% to 5% ionic liquid in polyurethane.

In the example embodiment, the membrane 3c of the reference electrode 3 comprises 2% ionic liquid in polyurethane.

In the example embodiment, the ionic liquid in the membrane 3c of the reference electrode 3 is selected from the group containing 1-ethyl-3-methylimidazolium bis(trifluoromethylsulfonyl)imide, 1-butyl-3-methylimidazolium hexafluorophosphate, 1-butyl-3-methylimidazolium acetate, 1-butyl-3-methylimidazolium chloride, choline acetate and/or choline phosphate.

In the example embodiment, the membrane 3c of the reference electrode 3 contains unplasticized polyurethane.

In the example embodiment, the reference electrode 3 and the indicator electrode 2 are in the form of a wire, which means that the tip of the electrodes intended to be inserted into the tissue is blunt, not sharply pointed.

In another example embodiment, the reference electrode 3 and the indicator electrode 2 are in the form of a needle, which means that the tip of the electrodes intended to be inserted into the tissue is pointed.

[FIG. 5] shows a probe 11 according to the present invention for real-time continuous measurement of the pH of animal tissues. The probe 11 comprises a body 12 in the form of a mounting capsule, which houses an electrode array 1, a vacuum system 13, a temperature sensor 14, and a signal cable 16. The electrode array 1 comprises the indicator electrode 2 and the reference electrode 3, which is all solid state and comprises a steel wire 3a with an applied layer of conductive polymer 3b and a membrane 3c containing ionic liquid and polyurethane. The vacuum system 13 is designed to attach the probe 11 to the tissue surface and comprises a vacuum tube 13a and a vacuum surface 13b. The temperature sensor 14 is a contact sensor designed to be applied to the outer surface of the tissue.

In the example embodiment, the electrode array 1 comprised in the probe 11 according to the present invention comprises the indicator electrode 2 and the reference electrode 3. The indicator electrode 2 is a wire of SS 316L surgical steel with a deposited antimony layer. In another example embodiment, the indicator electrode 2 is a SS 316L surgical steel wire with a deposited polymer membrane layer comprising a pH-sensitive ionophore on a conductive polymer layer. The indicator electrode 2 has a diameter of 0.5 mm and is in the form of a needle. Either the antimony layer or the polymer membrane layer, depending on the variant embodiment, is deposited using the galvanostatic technique. The reference electrode 3 is a wire 3a of SS 316L surgical steel with a deposited layer of conductive polymer 3b, which is PEDOT: PSS [poly(3,4-ethylenedioxythiophene)-poly(styrenesulfonate)] and with a membrane 3c containing the ionic liquid bis(trifluoromethylsulfonyl)imide 1-ethyl-3-methylimidazolium (4 mg) and polyurethane (196 mg). Polyurethane is an unplasticized polyurethane. The reference electrode 3 has a diameter of 0.5 mm and is in the form of a needle. A layer of conductive polymer 3b and ionic liquid membrane 3c is deposited using an electropolymerization technique.

In the example embodiment, the temperature sensor 14 is a PT-1000 temperature sensor.

In the example embodiment, the probe 11 at its periphery at the point of adhesion to the tissue comprises a suction cup envelope 15.

In the example embodiment, the body 12 of the probe 11 is made of a medical biocompatible material selected from medical silicones, acrylonitrile-butadiene-styrene terpolymer (ABS), HDPE, LDPE, polypropylene, PETG, polycarbonate, polyester, Kydex, POM, acetal copolymer, Delrin®, PET-P, Fluorosint®, Halar®, Hydex (PBT-P), Kynar, Noryl, Nylon, PEEK, polyethylenes (including LDPE, HDPE, and UHMW), polypropylene homopolymer, PPSU, PSU, Radel® A, Radel® R, Rulon 641, composites and mixtures thereof. Preferably, the body 12 of the probe 11 is made of acrylonitrile-butadiene-styrene (ABS) terpolymer.

In the example embodiment, the probe 11 has a cylindrical shape. The cylindrical shape of the probe ensures safety for the tissue undergoing pH measurement as well as for the surrounding tissue. The lack of sharp edges makes it impossible to accidentally cut into the tissue, break the surface or otherwise damage it.

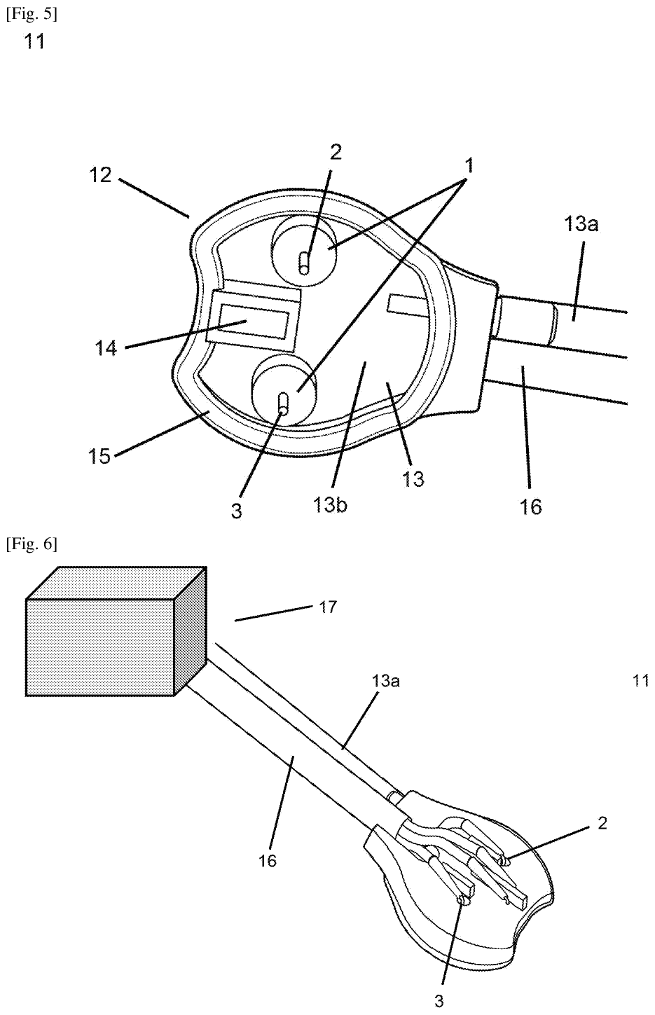

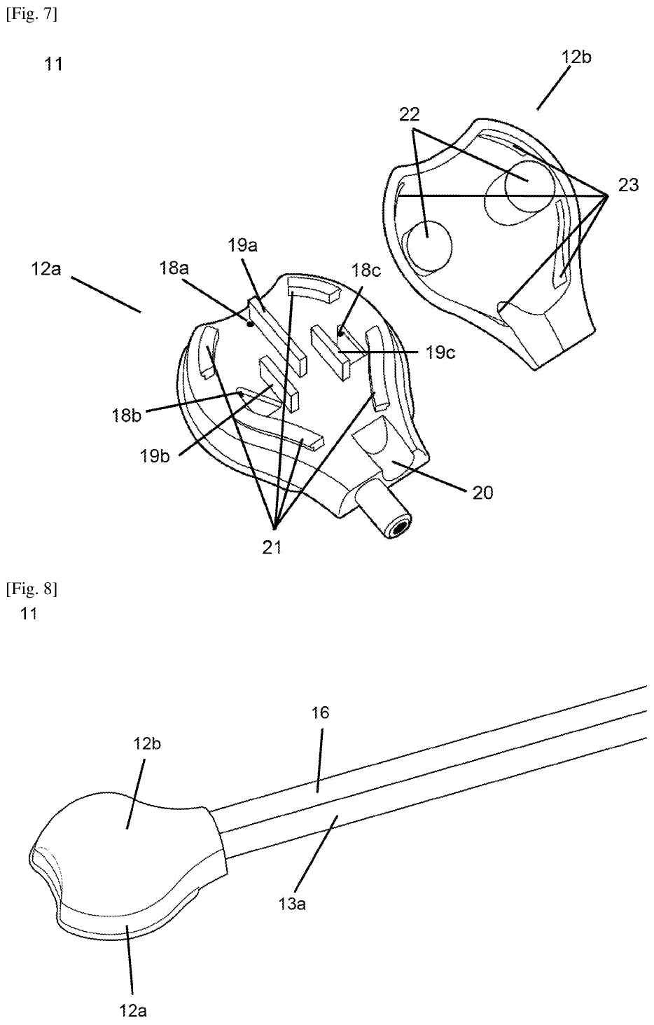

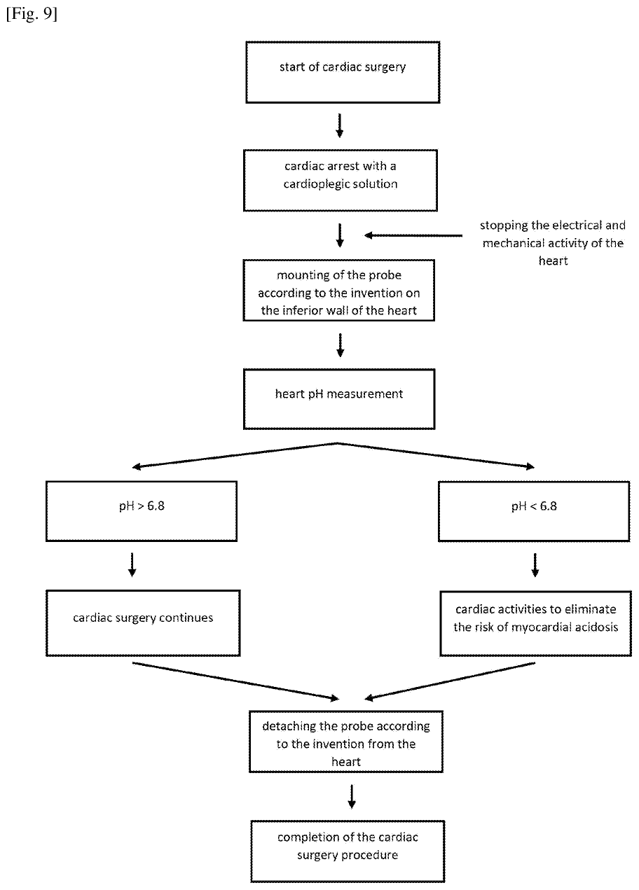

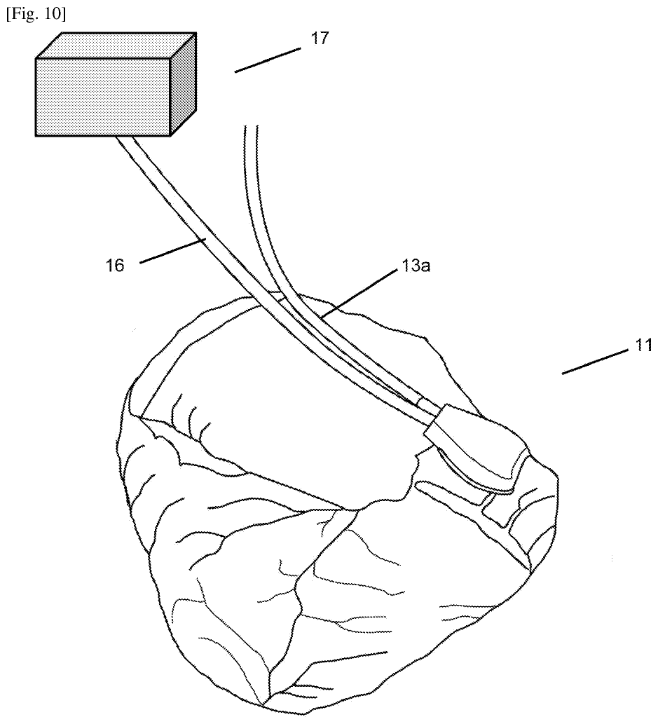

In the example embodiment, the height of the probe 11 is 6 mm. The height is understood to be the distance between the edge defining the suction surface (the bottom surface of the lower part of the body 12) and the edge defining the top surface of the upper part of the body 12. The justification for such dimensions of the probe (in the case of cardiac surgery) is that the optimal place for the application of the probe in the case of cardiac surgery is the bottom wall of the heart, at the confluence of the arterial vasculature of the right and left coronary arteries. This region of the heart rests against the pericardial sac from the posterior or diaphragmatic side during surgery. Thus, the probe is, as it were, pressed by the heart by its own weight. A significant probe height would cause the heart to crumple, make it impossible to manipulate the heart during surgery and increase the risk of perforation of the heart or surrounding tissues. A probe height of 6 mm (or approximately 6 mm) does not result in significant recessing of the arrested heart (treated with cardioplegia) when the probe is resting against the pericardial sac on the diaphragmatic side. And when the probe is resting against the free posterior wall of the pericardial sac, such a probe height results in free, safe deformation of the pericardial sac.

[FIG. 6] shows the probe 11 according to the present invention for real-time continuous measurement of the pH of animal tissues. In [FIG. 6], the signal cable 16 and vacuum line 13a are visualized. The signal cable 16 connects the indicator electrode 2 and the reference electrode 3 to a display device 17 of the measurement result, e.g. a cardiac monitor. The display device 17 simultaneously acts as a converter of the potential difference value measured by the electrode array according to the present invention into pH scale values. The signal cable 16 is additionally connected to the temperature sensor 14 (not shown in [FIG. 6]). Preferably, the signal cable 16 is a four-wire cable. The connection of the electrical components is performed in a manner known in the field, i.e. using pins, terminals, connectors, etc. The indicator electrode 2, the reference electrode 3, and the PT-1000 temperature sensor 14 are connected in a manner known in the field.

[FIG. 7] shows the probe 11 according to the present invention in an unfolded state with the division into a lower body part 12a and an upper body part 12b. In the example embodiment, the lower body part 12a comprises a passage 18a and a cable path 19a for the temperature sensor 14, a passage 18b and a cable path 19b for the indicator electrode 2, and a passage 18c and a cable path 19c for the reference electrode 3, a lead-out 20 of the signal cable and snap tabs 21. The upper body part 12b comprises a cable clamp 22 and latch grooves 23. The latch tabs 21 of the lower part 12a and the latch grooves 23 of the upper part 12b form a latching mechanism. Thus, the latching mechanism works in such a way that when the lower part 12a and the upper part 12b are folded and both parts are pressed together, the latch tabs 21 of the lower part 12a and the latch grooves 23 of the upper part 12b interlock. The latching mechanism allows the lower body part 12a and the upper body part 12b to be folded together without the need for additional bonding.

[FIG. 8] shows the probe 11 according to the present invention in an assembled state. [FIG. 8] shows the lower body part 12a, the upper body part 12b, the signal tube 16, and the vacuum tube 13a.

[FIG. 9] shows a schematic diagram of the method of protection and subsequent detection of acidification in human heart muscle.

During cardiac surgery using the described probe, the heart is arrested with a cardioplegic solution injected into the coronary vessels. At the stage of administration of the cardioplegic solution into the coronary vessels, the electrical and mechanical function of the heart is arrested. At this stage, the probe 11 is applied to the inferior wall of the heart, in the region representing the termination of the basin of the right and left coronary arteries. The probe 11 starts measuring the pH of the heart tissue while the surgical part of the procedure is performed according to the surgical plan. If the pH falls below the alarm value (pH=6.8) or drops sharply, the surgical team takes steps to eliminate the risk of myocardial acidemia. These actions include, for example, administering another dose of cardioplegia solution, achieving complete arrest of coronary blood flow, lowering the heart temperature, or administering cardioplegia solution by an alternative route. At the stage of completion of the surgical measures requiring the conditions of the arrested heart, just before the procedure to restore coronary artery blood flow and the expected return of electrical and mechanical function of the heart, the probe will be detached from the heart.

[FIG. 10] shows a schematic representation of the probe 11 according to the present invention attached to the surface of the heart while monitoring the pH value of the tissue.

In the example embodiment, the probe 11 is attached to the inferior wall of the heart, in the region of the posterior descending branch, at the junction of the blood supply of the right and left coronary arteries. The pH probe is attached to the surface of the heart using a vacuum mechanism between minus 100 and minus 400 mmHg. The vacuum is generated by an electric suction and delivered through a vacuum tube 13a. The pH measurement is transmitted via signal cable 16 to a display device 17, such as a cardiac monitor.

The probe 11 according to the present invention can be used to monitor the occurrence of acidemia of tissues such as muscle, connective tissue, parenchymal organ tissues, skin (also in the process of monitoring wound healing), as well as body parts or organs undergoing replantation or transplantation.

The calibration of the probe 11 is performed in such a way that the probe 11 is connected via a signal cable to a display device 17 of the measurement result, e.g. a cardio monitor. Then, the probe 11 (after removing the cover protecting the electrode array) is immersed in a tank of calibration solution 24 which is an electrolyte, preferably in a phosphate buffer with a pH close to the physiological pH of the tissue, i.e. pH=7. The calibration is performed automatically by the patient monitor. The duration of the calibration, i.e. the conditioning of the electrodes, can be adjusted as required, preferably 1 h. The probe 11 is then removed from the electrolyte reservoir and the device is ready for application to the tissue. The electrolyte reservoir is removed.

### INDUSTRIAL APPLICABILITY

The probe 11 according to the present invention can thus be used for many medical procedures, in particular surgical procedures. For example, the probe 11 can be used in cardiac surgery (e.g. monitoring the pH of cardiac tissue), vascular surgery (e.g. monitoring the pH of muscle tissue in patients with acute ischemia of the lower or upper limbs, in the event of acute aortic dissection) or transplantology (e.g. monitoring the pH of the organs, in particular the heart, intended for transplantation for their proper protection, i.e. adequate stopping of metabolism during transport).

All examples embodiments of the electrode array 1 and the probe 11 also refer to the pH measurement assembly 111 comprising the probe 11 together with the electrode array 1, the display device 17, and calibration solution 24, described in the description above.

The probe 11 according to the present invention can be used to monitor the pH of mammalian tissues, in particular those of primates. An example of which is a human. Hence, the probe 11 according to the present invention can be used to measure the pH of animal tissue, in particular human tissue. Human tissue is a preferred embodiment.

The electrode array 1 according to the present invention as such serves to measure the electromotive force and consequently, the pH, so it can also be used, inter alia, for the measurement of all samples characterized by small volumes/sizes. In particular, it is possible to measure food samples or liquid samples such as tap water.

Furthermore, it is to be understood that the applications of the invention indicated and described above are only given by way of an example. All example embodiments and their variants are given here only as non-limiting indications of the invention and may in no way limit the scope of protection, which is defined by the following claims. It should be understood that any technical solution used in the electrode array according to the present invention and/or in the probe according to the present invention can be carried out using equivalent technologies without exceeding the scope of protection.

Moreover, the use of the singular form when referring to individual elements of the invention also includes the plural form thereof and vice versa, unless the context indicates otherwise.

1. An electrode array (1) for measuring the pH of animal tissues comprising an indicator electrode (2), and a reference electrode (3), characterized in that the indicator electrode (2) is a steel wire with an applied layer of a pH-sensitive substance, the reference electrode (3) is a steel wire (3a) with an applied layer of conductive polymer (3b) on which a polymer membrane (3c) comprising a homogenic mixture of ionic liquid and polyurethane is applied.

2. The electrode array according to claim 1, characterized in that the indicator electrode (2) is an antimony electrode or a ruthenium(IV) oxide electrode.

3. The electrode array according to claim 1, characterized in that the indicator electrode (2) is the wire with an applied layer of antimony using the galvanostatic technique.

4. (canceled)

5. The electrode array according to claim 1, characterized in that the conductive polymer (3b) in the reference electrode (3) is PEDOT: PSS [poly(3,4-ethylenedioxythiophene)-poly(styrenesulfonate)], polypyrrole, or polyaniline.

6. (canceled)

7. The electrode array according to claim 1, characterized in that the conductive polymer (3b) in the reference electrode (3) is applied to the steel wire (3a) by electropolymerization or a ready-made suspension with the conductive polymer (3b).

8. The electrode array according to any of claim 1, characterized in that the polymer membrane (3c) contains from 1% to 5% ionic liquid in polyurethane.

9. (canceled)

10. The electrode array according to claim 1, characterized in that the ionic liquid in the polymer membrane (3c) is selected from the group consisting of 1-ethyl-3-methylimidazolium bis(trifluoromethylsulfonyl)imide, 1-butyl-3-methylimidazolium hexafluorophosphate, 1-butyl-3-methylimidazolium acetate, 1-butyl-3-methylimidazolium chloride, choline acetate and/or choline phosphate.

11. (canceled)

12. The electrode array according to claim 1, characterized in that the polymer membrane (3c) contains 4 mg of 1-ethyl-3-methylimidazolium bis(trifluoromethylsulfonyl)imide and 196 mg of polyurethane.

13. (canceled)

14. The electrode array according to claim 1, characterized in that the reference electrode (3) and the indicator electrode (2) are in the form of a wire or a needle.

15. The electrode array according claim 1, characterized in that the reference electrode (3) and the indicator electrode (2) have a diameter in the range of 0.2 to 1.5 mm.

16. The electrode array according to claim 1, characterized in that the reference electrode (3) and the indicator electrode (2) have a diameter of 0.5 mm.

17. A probe (11) for real-time continuous pH measurement of animal tissues comprising:
a body (12) in the form of a mounting capsule in which an electrode array (1) according to claim 1, a vacuum system (13), and a temperature sensor (14) are housed,
the vacuum system (13) configured to attach the probe (11) to the tissue surface and comprising a vacuum tube (13a) and a vacuum surface (13b), and
the temperature sensor (14) is a contact sensor configured to be applied to the surface of the tissue.

18. The probe (11) according to claim 17, characterized in that the temperature sensor (14) is configured to measure a temperature on the external surface of the tissue.

19. The probe (11) according to claim 17, characterized in that the probe (11) at its periphery at the point of adhesion to the tissue comprises a suction cup envelope (15).

20. The probe (11) according to claim 17, characterized in that the probe comprises a signal cable (16) configured to receive an electric signal from the indicator electrode (2), the reference electrode (3), and the temperature sensor (14) and to transmit the received signal to the display device (17) of the pH measurement result.

21. (canceled)

22. The probe (11) according to claim 17, characterized in that the body (12) of the probe (11) is made of a material selected from medical silicones, acrylonitrile-butadiene-styrene terpolymer (ABS), HDPE, LDPE, polypropylene, PETG, polycarbonate, polyester, acrylic composite of polyvinyl chloride, POM, acetal copolymer, PET-P polytetrafluoroethylene, ethylene chlorotrifluoroethylene copolymer (PBT-P), polyamide, PEEK, polyethylenes (including LDPE, HDPE, and UHMW), polypropylene homopolymer, PPSU, PSU, polyphenylsulfone, and composites and mixtures thereof.

23. (canceled)

24. The probe (11) according to claim 17, characterized in that the probe (11) has a lower body part (12a) and an upper body part (12b), the lower body part (12a) comprising a passage (18a) and a cable path (19a) for the temperature sensor (14), a passage (18b) and a cable path (19b) for the indicator electrode (2) and a passage (18c) and a cable path (19c) for the reference electrode (3), a lead-through (20) for the signal cable (16) and latch tabs (21) and the upper body part (12b) comprising a clamp (22) for the signal cable (16) and latch grooves (23), the latch tabs (21) of the lower part (12a) and the latch grooves (23) of the upper part (12b) being the latching mechanism.

25. (canceled)

26. An assembly (111) for measuring pH comprising a probe (11) according to claim 17, a display device (17), and a calibration solution (24).
